(19) United States
(12) Patent Application Publication
(10) Pub. No.: US 2012/0233718 A1
(43) Pub. Date: Sep. 13, 2012

(54) USE OF PON GENE CLUSTER IN PREPARING MEDICAMENT FOR TREATING ATHEROSCLEROSIS

(76) Inventors: Depei Liu, Beijing (CN); Zhigang She, Beijing (CN); Wei Zheng, Beijing (CN); Yusheng Wei, Beijing (CN); Houzao Chen, Beijing (CN)

(21) Appl. No.: 13/260,713

(22) PCT Filed: Mar. 18, 2010
(86) PCT No.: PCT/CN2010/071116

§ 371 Date: Dec. 16, 2011

(30) Foreign Application Priority Data
Mar. 27, 2009 (CN) 2009 10081009.9

(51) Int. Cl. A01K 67/027; C12N 15/55; C12N 15/89

(52) U.S. Cl. 800/22; 800/24; 536/23.2

(57) ABSTRACT

Use of PON gene cluster in preparing medicament for treating atherosclerosis in mammals, wherein the PON gene cluster treat atherosclerosis by promoting stability of atherosclerotic plaque. Method for the developing PON gene cluster transgenic mouse model and use of PON gene cluster in the development of PON gene cluster positive transgenic mouse model with atherosclerosis are also provided.

### FIELD OF THE INVENTION

The present invention relates to the use of Paraoxonase Gene Cluster in preparing medicament for promoting the stability of atherosclerotic plaques. Furthermore, the present invention relates to the method for developing PON Gene Cluster transgenic mice models and the use of PON Gene Cluster in the developed PON Gene Cluster positive transgenic mice models with atherosclerosis.

### BACKGROUND OF THE INVENTION

1. Atherosclerosis and Plaque Rupture

Cardiovascular diseases are the major lethal and pathogenic elements in our country and the developed country. Atherosclerosis is a primary element for causing the cardiovascular diseases (Libby, 2002) (Glass and Witztum, 2001). In the present, atherosclerotic plaque rupture and the subsequent thrombosis, rather than blood vessel stenosis resulted from atherosclerosis, are considered as major reasons for contributing to atherosclerosis-related symptoms of ischemic (especially myocardial infarction and stroke). For clinical treatment of atherosclerosis and the complication thereof, it is crucial to develop a therapic method aiming efficiently at the element resulting in the plaque rupture. Oxidized low density lipoprotein (oxLDL) plays a key role in plaque formation and aggravating inflammation (Libby, 2002; Steinberg, 1997). oxLDL can stimulate endothelial cell, etc. to express adherent factors, chemotactic factors, and other cytokines. Consequently, it mediates the adherence and recruitment of monocytes/macrophages to lower layer of endothelial cell, and differentiated into macrophages (Lusis, 2000). Furthermore, the recruited macrophages phagocytize and oxidize LDL. Excessive phagocytosis makes apoptosis and necrosis per se, and transforms the macrophages into foam cells. The transformed macrophages form a fatty streak and the center of the necrosis in the earlier stage. Furthermore, they become an actively inflammatory center due to a large amount of secreted inflammatory factors and matrix metalloproteinases (MMPs). The actively inflammatory center promotes the development of the plaque. Finally the plaque ruptures and the complication is initiated (Galis, 2004; Schwartz et al. 2007).

2. Association Between Paraoxonase (PON), oxLDL and Atherosclerosis

2.1 Introduction of Paraoxanase (PON) Family

Paraoxonase family is also named as PON family, which is a protease family controlling the hydrolysis of esters. Until now, it has been reported that the said family has three members, PON1, PON2 and PON3 (Primo-Parmo et al, 1996). Most of the studies are concerned on PON1 and PON2.

The human PON1 Gene contains 9 exons and 8 introns and encodes a protein consisting of 355 amino acids, having a relative molecular weight of approximate 43 kDa (Mackness et al., 1998). The studies on the structure of PON1 protein indicate that the PON1 protein consists of six-layer beta-helix, a unique active site and a His-His structure-based dissimilation center (Harel et al., 2004). The human PON1 Gene contains three cysteine residues, wherein a disulfide bond is formed between the position of 42 and 352, and the cysteine residue in the position of 284 is in a free status, which is necessary for optimizing the activities of paraoxonase and arylesterase. PON1 is synthesized in liver, then secreted into the blood and binds with HDL specifically. PON1 can hydrolyze aromatic esters substrates, for examples, phenylacetate, Phenyl Thioacetate and 2-Naphthalenol acetate. In addition, some kinds of aromatic lactone, fatty lactone and cyclic carbonate can also be hydrolyzed by PON1. Furthermore, PON1 can catalyze reverse reaction of esterification, hydrolysis (Mackness et al., 2002; Ng et al, 2001).

PON2 is a second member of Paraoxonase Gene family on chromosome. Similar to PON1, PON2 contains 9 exons and 8 introns. PON2 has 79%˜90% identity to PON1. However, PON2 does not exist on HDL, but exists on membrane lipoprotein. PON2 is widely expressed in the tissues of human liver, brain and kidney, etc. The paraoxonase activity and arylesterase activity of PON2 are weaker than those of PON1 (Ng et al., 2001).

POM 3 contains 5 extrons and 3 introns and encodes a protein consisting of 353 amino acids, having molecular weight of approximate 40 kDa. In human body, PON3 mainly exists on HDL particles. However, the concentration of PON3 is approximately 50 folds lower than that of PON1 (Draganov et al., 2000).

PON2 and PON3 have structural and functional similarities to PON1. PON2 is widely expressed in the tissues of mammals and is considered as an intracellular antioxidant to delay the oxidation of LDL. Similar to PON1, PON3 is also synthesized in liver and exerts its antioxidant functions by binding with HDL (Ng et al., 2005).

2.2 Association Between Members of PON Family and oxLDL

PON is paraoxonase. It has activity in catalyzing hydrolysis reactions and can degrade various kinds of esters produced by esterification. oxLDL is the production of esterification. That is to say, POD can resist the formation of oxLDL. Although the distributions of the three members of the PON family are distinct, their functions are similar. All of them have the paraoxonase activity and can catalyze reverse reaction of esterification, hydrolysis. Therefore, PON is one of important elements for resisting the oxLDL formation (Aviram and Rosenblat, 2004).

2.3 Effect of Members of PON1 Family on Atherosclerosis

2.3.1 Association Between PON1 and Atherosclerosis

PON1 exists on HDL particles, resists the oxidation of LDL, reduces oxLDL levels and has an anti-atherosclerotic effect (Watson et al., 1995). The anti-atherosclerotic effect of PON1 has been demonstrated in the experiments by using PON1 transgenic and Knockout animals.

The results of the experiments have demonstrated that the purified PON1 resists the oxidation damage of LDL and accelerates the degradation of phophoslipid hydroperoxide (Watson et al., 1995). Furthermore, it has been demonstrated that PON1 can hydrolyze the oxidized lipid in atherosclerotic plaques of human coronary artery (Hedrick et al., 2000). Meanwhile, the results in the experiments using PON1 overexpressing and knockout animals have also suggested the anti-atherosclerotic effect of PON1. Compared with the control mice, high-fat diet feeding resulted in more serious AS in PON1 Gene-knockout mice. Furthermore, the HDL in PON1 Gene-knockout mice also lost the effect on protecting LDL from being oxidized (Shih et al., 1998). However, human PON1 Gene overexpressing mice resisted the occurrence of AS under the same conditions (Tward et al., 2002).

Meanwhile, clinical experiments also provide the evidence for supporting the PON1 functions. The studies on SNPs of PON1 demonstrate that the low activity of PON1 in vivo will increase the incidence of atherosclerosis (Watzinger et al., 2002).

2.3.2 Association Between PON2 and Atherosclerosis

PON2 is widely expressed in the body of mammals. It is considered that PON2 has effect on resisting the oxidation of LDL in the cell (Ng et al., 2001). PON2 overexpressing cells can reduce the oxidation level of LDL and can more efficiently resist the oxidation stress induced by H₂O₂ and oxidized lipid (Ng et al, 2001). Meanwhile, the studies on PON2 knockout animals demonstrate that the knockout mice are more susceptible to the formation of atherosclerotic plaque than wild-type mice from the same brood (Ng et al., 2006). This also strongly supports that PON2 also has an anti-atherosclerotic effect.

Meanwhile, studies on SNPs of PON2 also suggest that PON2 is closely related with plasma total cholesterol concentration, mediation of glycerin trilaurate, kidney disease and type II diabetes (Hegele et al, 1997). Furthermore, the study on the association between PON2 and endarterium hyperplasia in familial hypercholesterolemia Caucasian also can explain the association between PON2 and the development of atherosclerosis (Leus et al., 2001).

2.3.3 Association Between PON3 and Atherosclerosis

PON3 is a protein synthesized in liver, having a molecular weight of 40 kDa. In the serum of human or rabbit, PON3 binds with HDL, rather than LDL. Furthermore, the concentration of PON3 on HDL is 50 folds lower than that of PON1 (Draganov et al., 2000). Some studies suggest that Human Artery Endothelial Cells pretreated with PON3 have lightly effect on resisting the production of oxLDL and can inactivate the produced oxLDL. However, the hydrolytic activity of PON3 is weaker than that of PON1. PON3 can not hydrolyze paraoxon phospholipid. In HepG2 cells and the mice livers stimulated with high-fat diet, the expression of PON3 is not regulated by the oxidized phospholipid. These demonstrate that the anti-atherosclerotic effect of PON3 is weaker than that of PON1. However, PON3 still has some effect (Reddy et al. 2001). Some studies have indicated that in the ApoE knockout mice, an atherosclerosis-susceptible model, the expression of adenovirus-mediated PON3 resists the initiation of atherosclerosis (Ng et al., 2007). The PON3 transgenic mice also display an anti-atherosclerotic ability (Ship et al., 2007). Based on the above reasons, although the anti-atherosclerotic efficiencies of the three PON family members are distinct, all of them have an effect on resisting the initiation and development of atherosclerosis. However, the disclosed results simply focused on the effect of individual PON family member on minimizing the plaque areas of atherosclerosis. Until now, none of the studies have demonstrated the effects of PON family as a gene cluster on inhibiting the development of plaque and further stabilizing the atherosclerotic plaque.

### SUMMARY OF THE INVENTION

As described above, the studies on individual PON1, PON2 and PON3 genes have suggested that these genes can inhibit atherosclerosis by inhibiting the oxidation of LDL. The present invention relates to the effect of PON as a gene cluster on atherosclerosis.

Therefore, the first aspect of the invention relates to the use of PON gene cluster in preparing the medicament for treating atherosclerosis in mammals. Preferably, the said mammal is selected from mouse or human. More preferably, the said mammal is human.

The second aspect of the invention relates to a method for the developing a PON gene cluster transgenic animal model, comprising the following steps:

a) A vector comprising PON gene cluster was linearized with an appropriate restrictive endonuclease. The vector DNA was taken out and treated by conventional method to be used for microinjection.

b) The said DNA was diluted to approximate 1-2 ng/μL with buffer for microinjection and then micro-injected into the fertilized egg of the animal surrogate.

c) The fertilized egg was placed in M16 medium after being micro-injected, incubated at 37° C. for 1-2 days.

d) The fertilized egg of step c) was transferred to pseudo-pregnant animal surrogate. After the newborn animals were delivered, PON gene cluster positive animals were selected by PCR and Southern Blot Analysis.

Preferably, the vector is BAC vector RP11-104H16, and the restrictive endonuclease is Not I. Preferably, the said animal is mice. Preferably, the said fertilized egg is C57BL/6J fertilized egg.

The third aspect of the invention relates to the use of PON gene cluster in the development of PON gene cluster positive transgenic mice models with atherosclerosis.

Preferably, the said transgenic mice models were obtained from the following steps:

a) Both PON gene cluster positive and apoE^{+/-} mice were obtained by crossing the mice obtained according to claim 7 and the apoE^{-/-} mice with atherosclerosis.

b) The mice obtained from a) were further crossed with apoE^{-/-} mice for another generation and the mice with genotype both of PON gene cluster positive and apoE^{-/-} were obtained. i.e., PON gene cluster positive transgenic mice models with atherosclerosis were obtained.

c) The mice obtained from b) were continuously crossed with apoE^{-/-} mice to obtain large number of PON gene cluster positive transgenic mice models with atherosclerosis.

In other words, the PON gene cluster construction transgenic mice comprising all of the three PON gene sequences and their corresponding regulatory sequences were selected in the present invention. Furthermore, the said transgenic mice were crossed with conventional atherosclerotic mice models with apoE gene knockdown. Therefore, both PON gene cluster positive and apoE gene deficient mice models were obtained and atherosclerosis was studied using these models. The mechanism of PON gene cluster in macrophages systems underlying atherosclerosis was studied by extracting macrophages from the abdomen of the transgenic mice. It was found that on the contrary to the mechanisms of PON1, PON2 and PON3, the PON gene cluster exerts a therapeutic effect on atherosclerosis via promoting the stability of atherosclerotic plaques. The unique mechanism of PON gene cluster provides the basis for developing a medicament for treating atherosclerosis by promoting the stability of atherosclerotic plaques.

### DESCRIPTION OF THE FIGURES

FIG. 1 displays a whole experimental scheme of the invention.

FIG. 2 displays genomic fragment structure containing PON gene cluster. The micro-injected fragment is a human genomic DNA with the length of 170 kb containing PON1, PON2 and PON3 structural gene (shaded rectangle) and lateral sequences (blank rectangle).

FIG. 3 displays the identification of BAC clone 04H16 by PCR method with 4 pairs of primers.

FIG. 4 displays the PFGE drawings of BAC-RP11-104H16. The molecular weight standards are derived from Molecular Weight Standard N0350 of some PFG fragments of NEB.

FIG. 5 displays the identification of transgenic mice by PCR. P1-P5: transgenic mice; WT: wildtype; BAC: RP11-104h16; DL2000: Molecular Weight Standard.

FIG. 6 displays the identification of transgenic mice by Southern Blot. The DNAs for Southern Blot analysis were obtained from the following: The first lane, human genomic DNA; The second lane, PC transgenic mice of P1 strain; The third lane, PC transgenic mice of P2 strain; The fourth lane, PC transgenic mice of P3 strain; The fifth lane, PC transgenic mice of P4 strain; The sixth lane, PC transgenic mice of P5 strain; The seventh lane, wildtype mice. All of DNAs were digested with EcoRI and hybridized with three pairs of primers, respectively. The lower primer is corresponding to human PON1 gene sequences (the position 10090 to 10515 of human BAC clone RP11-104H16); The middle primer is corresponding to human PON3 gene sequences (position 96196 to 96727 of human BAC clone RP11-104H16); And the upper primer is corresponding to human PON2 gene sequences (position 165366 to 165663 of human genome).

FIG. 7 displays the expressing profiles of human (H) PON1, PON2 and PON3 in the tissues, comprising Heart (Ht), Kidney (Kd), Liver (Li), Lung (Lu), Muscle (Ms), Intestine (In), Spleen (Sp), Stomach (St), Aorta (Ao), Ovary (Ov), and Brain (Br), of transgenic mice. The endogenous (M) PON1, PON2, PON3 and actin of mice were used as control.

FIG. 8 displays the expressions of H PON were absent in the organs of wildtype mice.

FIG. 9 displays that among the five strains of mice, the liver of P2 strain of transgenic mice shows the highest expressing level of human PON1 gene.

FIG. 10 displays the expressing profiles of human PON1, PON2 and PON3 protein in Liver and Aorta of P2 strain of transgenic mice.

FIG. 11 displays the expressing profile of PON1 gene in HDL of PC transgenic mice.

FIG. 12 displays the detection of relative paraoxonase activity of HDL in fasting wildtype mice (pallid column) and PC transgenic mice (dark color column) using the kits for detecting paraoxonase activity. The average for each genotype was displayed (n=10). *p<0.05.

FIG. 13 displays the reduced area of plaque and lipid in PC transgenic/ApoE^{-/-} mice compared with ApoE^{-/-} control mice. The results of AS plaque of ApoE^{-/-} mice stained with HE were displayed. The area of plaque in PC transgenic/ApoE^{-/-} mice (B) was 30.8% smaller than that in control group of ApoE^{-/-} mice (A) (C). The area of lipid core of the plaque in PC transgenic/ApoE^{-/-} mice (B) (the black region of the plaque in the figure) is 13.1% smaller than that in control group of ApoE^{-/-} mice (D). *p<0.05, **p<0.01, n=10 for each group.

FIG. 14 displays that the plaque in PC transgenic/ApoE^{-/-} mice is more stable than that in control group of ApoE^{-/-} mice. The isolated aortic sinus was stained with oil red for staining lipid (A & B), with trinitrophenol and Sirius for staining collagen (C & D), with SMA for staining SMC (E & F) and with Moma-2 for staining macrophages (G & H). Compared with control group of ApoE^{-/-} mice, PC transgenic/ApoE^{-/-} mice displayed an increased percentage of collagen (76.9% increased), SMA (15.8% increased), macrophages (22.3%) and a decreased area of lipid (9.5%). *p<0.05, **p<0.01. n=10 for each group. J: The score of the stability of the plaque in PC transgenic/ApoE^{-/-} mice is 70% higher than that in ApoE^{-/-} mice.

### DETAILED EMBODIMENTS

The following examples are intended for the further understanding of the present invention. The examples are used to illustrate the invention, but not to limit the protecting scope of the present invention. It is obvious to make modification and alteration of the invention without detaching the subject matter of the invention. Therefore, these modifications and alterations are fallen into the protecting scope of the present invention.

### EXAMPLES

### Example 1

### Methods and Materials for the Study

A Bacterial Artificial Chromosome (BAC) for Transgenes

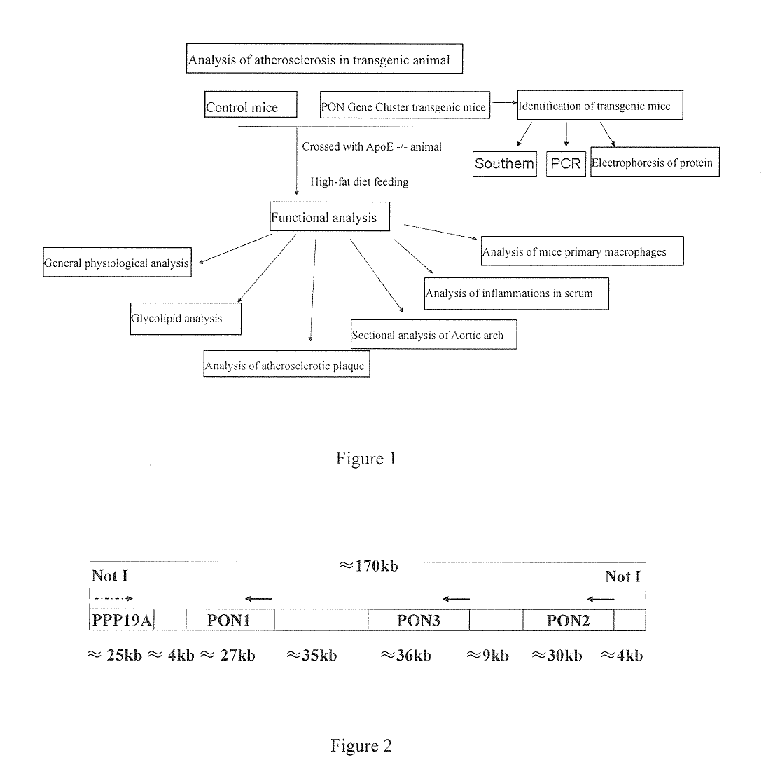

The BAC vector comprising human PON gene cluster (RP11-104H16) was purchased from Chori BacPac. The said clone has total length of 170 kb and comprises human PON1, PON2, PON3 structural gene and their corresponding lateral sequences, as shown in FIG. 2. The BAC was confirmed by PCR, PFGE and the Internet Search.

Experimental Mice and Diets

C57BL/6 mice, F1 Hybrid of C57BL/6 and FVB male mice, and the diets for the mice were provided by the Animal Center of Academy of Chinese Military Medical Sciences. The mice were bred in animal house of 2 grades, with free access to clear water and diets except where indicated otherwise. 12 hour light cycle period was adopted, with lighting from 7 am to 7 pm and dark from 7 pm to the next 7 am. All of the animal experiments were performed based on the Animal Care and Regulation of Academy of Chinese Medical Sciences. The high-fat diets for inducing atherosclerosis were provided by the Animal Center of Academy of Chinese Medical Sciences. The components of per 10 kg of the high-fat diets are indicated as follows: basic diets 8875 g, triglyceride 1000 g, and cholesterol 125 g.

Construction of PON Gene Cluster Transgenic Mice

BAC DNA was digested with Not I and a linearized vector was obtained. The linearized vector was treated by a conventional method and was used for microinjection (Gao et al., 2005). The complete DNA was diluted to the concentration of 1.2 ng/μL and was micro-injected into the fertilized eggs of C57BL/6J mice to construct PON gene cluster transgenic mice.

Morphologic Analysis of the Tissue and Estimation of the Stability of the Plaque

Mice were decapitated after 16-week high-fat diets feeding. Systematic perfusion was performed through left ventricle using cold PBS and 4% paraformaldehyde solution. The hearts with ascending aortas were collected (10 per group) and were embedded with OTC. The continuous frozen sections were sliced from the root of the aorta with the thickness of 10 μm. Aortic valve was used as a marker for position (Ni et al, 2001). Five continuous sections, spaced 80 μm apart were analyzed as staining indicators. The obtained slices were stained with H & E and the morphology was analyzed. The lipid core and the collagens were stained with oil red O and trinitrophenol and Sirius, respectively. The SMA and the macrophages were stained with anti-α-smooth muscle cell (SMC)-ACtin (Abeam, ab5694) antibody and anti-MOMA-2 (Serotec, MCA519G) antibody, respectively, by immunohistochemical technology. The corresponding staining regions were scanned and the images were quantitatively analyzed by Imagepro Plus 5 Software. The stability of the plaque was analyzed by estimating the percentages of the main components of the plaque, lipid core, collagen tissue, smooth muscle cells and macrophages. The total stability was indicated as a score of the stability of the plaque. Score of the stability of the plaque=(area of SMC+area of collagen)/(area of macrophages+area of lipid core) (Ni et al, 2001).

Statistical Analysis

All of the values were represented as mean±standard deviation. Student's t test was used to analyze the difference between the two groups. P<0.05 was deemed as significant difference.

### Example 2

### Results of the Study

BAC RP11-104H16 Contains Complete Human PON Gene Cluster Elements

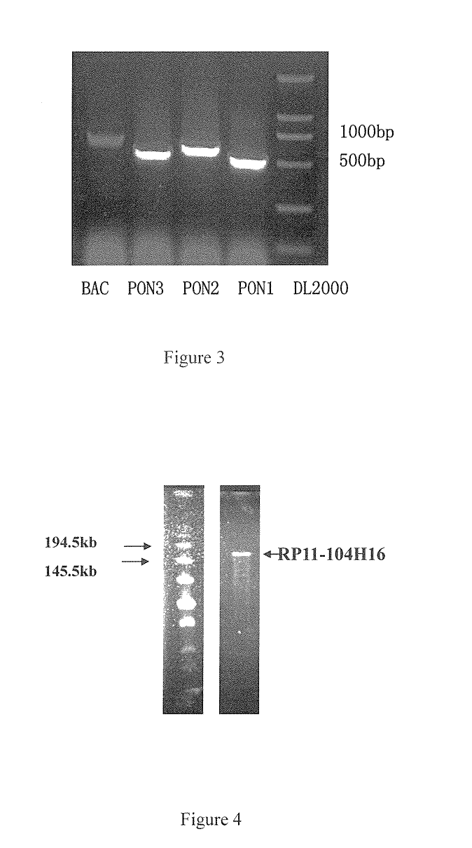

The correctness and the integrity of BAC RP11-104H16 used for microinjection were confirmed by several PCR experiments against different sites (Refer to FIG. 3).

The size of BAC RP11-104H16 was identified as approximate 170 kb, as expected previously (Refer to FIG. 4).

Preparation of DNA Used for Microinjection

The obtained highly qualitative and highly purified DNA can be used for microinjection. The concentration of DNA solution is approximately 25-30 ng/μl, which was diluted with buffer for microinjection to 1-2 ng/μl. The diluted solutions were divided into 20 μl per tube and stored at −20°C to be used for microinjection.

The microinjected fertilized eggs were placed in M16 medium after the being microinjected and incubated at 37° C with CO₂ for 1-2 days. The rate of binary fission was more than 90% and the rate of ternary fission was more than 40%. These results suggested that the quality of the DNA was suitable for being micro-injected into the fertilized eggs to prepare the transgenic mice.

Construction of Human PON Cluster Transgenic Mouse Strain

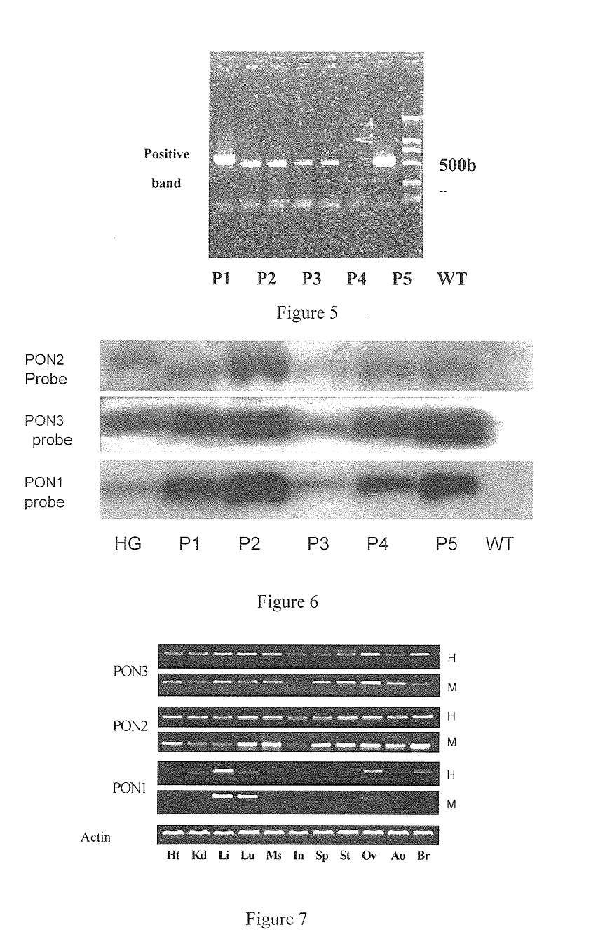

The purified linear DNA comprising PON gene cluster was microinjected into the arsenoblast of fertilized eggs of C57BL/6 mice. The said fertilized eggs were then transferred to pseudo-pregnant mice. The said mice were gestated and delivered 58 newborn mice. The ears of the newborn mice were punched and the tissues were digested. The positive transgenic mice were screened by the method of PCR. The primers were designed to be complementary to the sequences of the transferred human PON gene cluster, but not be complementary to the endogenous sequences of the mice. Therefore, positive bands can be amplified by the said primers when the genome of transgenic mice was used as template, whereas no amplified products can be observed when the genome of wildtype mice was used as template. The positive transgenic mice can be identified by the primers. The positive fragments were amplified in five newborn mice, and named as P1, P2, P3, P4 and P5, respectively (Refer to FIG. 5).

Five strains of positive transgenic mice were furthermore identified by Southern Blot. The tails of the mice were cut off and genomic DNA were isolated. The genome was digested by EcoRI, transferred to the filter and hybridized. The sequences of the probes P1, P3 and P2 were corresponding to the sequences of transgenic human PON1, PON3 and PON2, respectively. The sequences display no obviously homological to the endogenous sequence of mice after alignment in BLAST. The whole transgenic products with the size of 2 kb, 5 kb and 7 kb were obtained after the hybridization with the probes. Whereas no products were obtained in the normal genome of the mice. The experimental results show that the sizes of the hybridized bands were the same as expected and demonstrate that all of the five mice were transgenic positive mice.

The Correct Expression of the Transgenic Gene in Mice Body

After the establishment of the transgenic mice, the tissue-specific expressions of the three members of transgenic PON gene cluster were furthermore detected in vivo of the mice. The expressed products in vivo of the mice were detected by RT-PCR. The tissues from the Heart (Ht), Kidney (Kd), Liver (lv), Muscle (Ms), Intestine (In), Spleen (Sp), Stomach (St), Ovary (Ov), Aorta (Ao) and Brain (Br) of the transgenic and negative mice were isolated. Total RNA was isolated and were synthesized into the first strand of cDNA by Reverse Transcription. 1 μl of cDNA product was used as template. The used primers were indicated in Table 1:

| Primers | Sequences 5′-3′ |
|---|---|
| hPON1-RT-S | AAAGGAATCGAAACTGGCTCTG |
| hPON1-RT-A | GACTGTTGGGGTTGAAGCTCT |
| hPON2-RT-S | CTCTTCGTGTATGACCCGAAC |
| hPON2-RT-A | ACCCATTGTTGGCATAAACTGTA |
| hPON3-RT-S | AACTTTGCGCCAGATGAACCA |
| hPON3-RT-A | TCATGTGGGGATGATTCACAAC |
| mPON1-RT-S | TACTGGTGGTAAACCATCCAGA |
| mPON1-RT-A | GCAGCTATATCGTTGATGCTAGG |
| mPON2-RT-S | GCTCTGAGTTTGCTGGGCAT |
| mPON2-RT-A | CCACGCTAAAGAAAGCCAGG |
| mPON3-RT-S | CCTCACTGGACTTCCGTCG |
| mPON3-RT-A | GGATCAACGGTCAAGTTATCCAC |
| β-actin-s | GTGGGGCGCCCCAGGCACCA |
| β-actin-a | CTCCTTAATGTCACGCACGATTTC |

The annealing temperature of the above primers were 60° C. The numbers of PCR cycles was 30, except that of β-actin was 24. The electrophoresis results of PCR products show that the distributions of the expressed three members of PON gene were corresponding to the three endogenous PON genes of mice. That is to say, PON 1 was mainly expressed in the liver, and PON2 and PON3 were expressed more widely (Refer to FIG. 7). NO human PON gene expression was detected in negative mice in the same brood (Refer to FIG. 8).

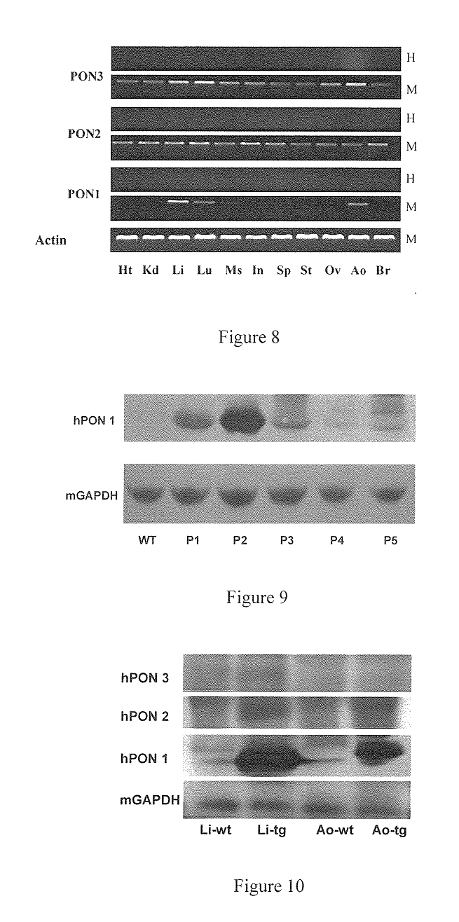

The livers derived from five strains of transgenic mice and negative mice from the same brood were homogenized and the proteins were isolated. The expression of human PON 1 was detected by Western Blot. The results indicate that among the liver derived from the five strains of transgenic mice, the expression of PON1 is the highest in the liver derived from P2 strain (Refer to FIG. 9).

The proteins isolated from the liver and aorta of P2 strain transgenic mice were detected by Western Blot. The results indicated that the liver derived from P2 transgenic mice displays the expression of human PON1, PON2 and PON3, whereas the aorta thereof mainly displays the expression of PON2. At the same time, a small amount of PON 1 and microscale of PON3 were also detected in the aorta. This may be due to the existence of HDL in the aorta (Refer to FIG. 10).

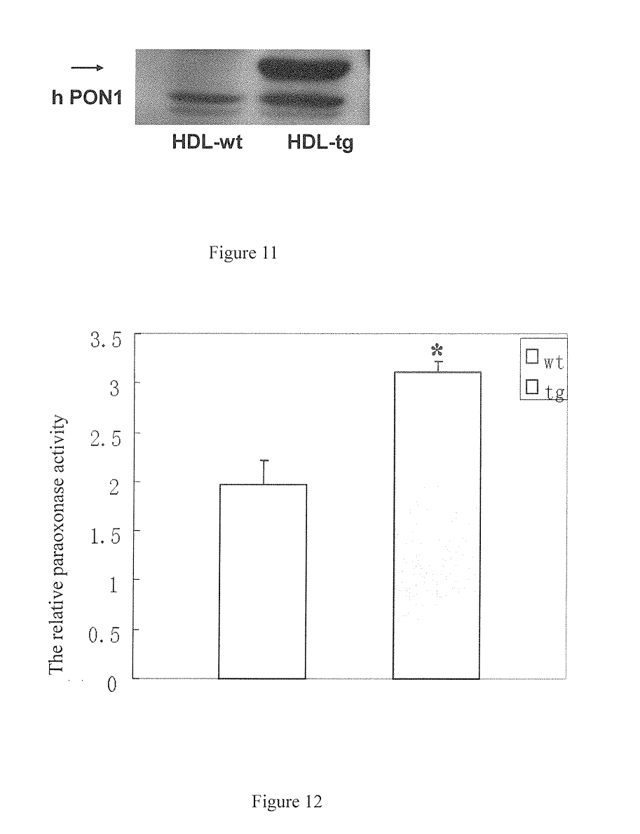

The serums derived from ten fasting P2 strains of transgenic mice and ten fasting control mice from the same brood were isolated and were ultra-centrifuged to isolate HDL. The serums from the two groups were equivalently mixed, respectively. After partially defatting treatment, the expressions of human PON proteins were detected by Western Blot. The results indicated that human PON 1 protein can only be detected on P2 transgenic mice. (Refer to FIG. 11).

The paraoxonase activity was measured in the other part of isolated HDL by using ethyl benzoate as substrate. The results indicated that the paraoxonase activity of P2-HDL was approximately 1.7 fold higher than that of control non-transgenic mice (Refer to FIG. 12).

Based on the above statement, the high levels of expression and the corresponding activity were achieved in vivo of the transgenic mice. Based on the experimental results, we selected the P1 and P2 strains of transgenic mice with high copies for further studies. The results from P2 strains were mainly indicated. The results from P1 strains were indicated only when they were distinct to those from P1.

No Obvious Phenotypes were Observed in Normal Transgenic Mice

The frequency of the exogenous gene in each strain of transgenic mice was approaching 50%, which was in consistent with mendel's law. These results indicated that the transferred gene did not have lethal effect. No obvious behavioral abnormal was observed in the transgenic mice. When provided with normal diets, the levels of the body weight, plasma total cholesterol (CHO), high density lipoprotein cholesterol (HDL-CHO), low density and very low density lipoprotein cholesterol (LDL/VLDL-CHO), triglyceride (TG), blood glucose in male and female transgenic mice were similar to those in control mice from the same brood (refer to table 2).

Construction of h PC⁺/apoE^{-/-} Mice Strain

The two strains of P1 and P2 mice were selected and crossed with atherosclerotic models of apoE^{-/-} mice. The effects of the transferred human PON gene cluster on the formation of atherosclerosis were studied. The mice with H PC⁺/apoE^{-/-} genotype were obtained after the first generation by crossing the positive mice and the apoE^{-/-} mice. The obtained H PC⁺/apoE^{-/-} mice were continuously crossed with apoE^{-/-} mice and consequently, enough numbers of H PC⁺/apoE^{-/-} mice and non-transgenic mice from the same brood were obtained. The atherosclerosis was induced by high-fat diet and the effect of transferred human PON gene cluster on atherosclerosis was observed. The apoE^{-/-} mice of same gender, derived from the same brood were used as control. The frequencies of various kinds of genotypes in the two strains of transgenic mice were in consistent with mendel's law. No significant difference was observed between the two strains of transgenic mice in all experiment, suggesting the results were common regulations rather than strain specific.

PON Gene Cluster Promotes the Stability of the Atherosclerotic Plaque

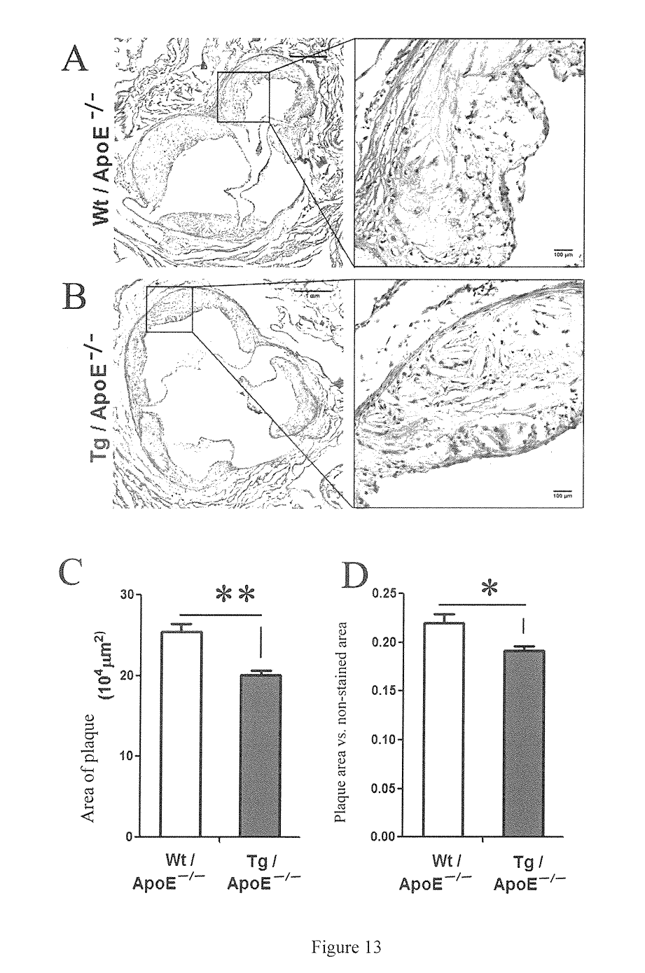

To study the effect of PON gene cluster on promoting the stability of atherosclerotic plaque, the hearts derived from female PC Tg/ApoE deficient and ApoE deficient mice treated with high-fat diets for 16 weeks were collected and analyzed by staining the sections. The results from H & E staining suggested the area of plaque in PC Tg/ApoE deficient mice was reduced approximately 30.8% compared with that in control mice (Refer to FIG. 13C). Furthermore, the ratio of non-stained area (indicative as necrotic core) to the total area of the plaque was also reduced approximately 13.1% compared with that in control mice (Refer to FIG. 13D).

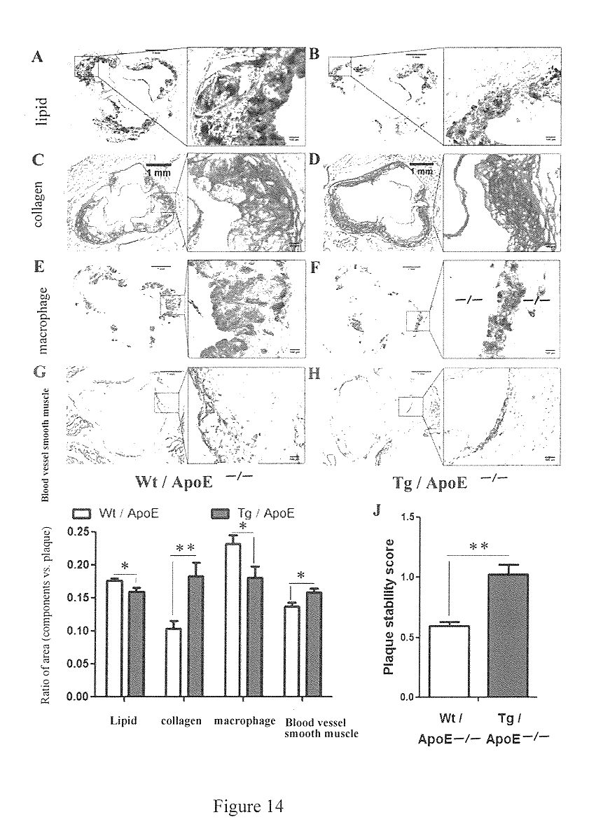

These results indicated that in one respect, PON gene cluster inhibited the formation of atherosclerosis. In the other respect, these results also suggested that the plaque in the PON gene cluster transgenic mice may have a thicker fibrous cap and a smaller necrotic core. Further estimation of the stability by comparing the components of the plaque suggested that the plaque in PC Tg/ApoE deficient mice contained more collagens (76.9%, refer to FIG. 14C, FIG. 14D and FIG. 14I) and smooth muscle cells (15.8%, refer to FIG. 14G, FIG. 14H and FIG. 14I), and less macrophage infiltrations (22.3%, refer to FIG. 14E, FIG. 14T and FIG. 14I) and lipid core (9.5%, refer to FIG. 14A, FIG. 14B and FIG. 14I). The said alterations in the components of the plaque suggested that the PON gene cluster promotes the stability of the atherosclerotic plaque. Correspondingly, the score of the stability was increased with the increase of the transferred PON gene cluster.

| | | | | | | |
|---|---|---|---|---|---|---|
| sample | total | HDL | LDL/VLDL | | | Body |
| numbers | cholesterol | cholesterol | cholesterol | Triglyceride | Glucose | weight |

| mouse | n | mg/dl | mg/dl | mg/dl | mg/dl | mg/dl | g |
|---|---|---|---|---|---|---|---|
| female, diets | | | | | | | |
| wildtype | 7 | 101 ± 4 | 90 ± 3 | 12 ± 1 | 110 ± 9 | 129 ± 9 | 22 ± 1 |
| PC Tg | 7 | 108 ± 5 | 95 ± 3 | 15 ± 1 | 105 ± 10 | 140 ± 14 | 22 ± 1 |
| male, diets | | | | | | | |
| wildtype | 9 | 102 ± 2 | 90 ± 3 | 11 ± 1 | 111 ± 9 | 126 ± 6 | 23 ± 1 |
| PC Tg/ | 10 | 104 ± 3 | 95 ± 4 | 14 ± 1 | 94 ± 7 | 145 ± 10 | 23 ± 1 |

### REFERENCES

Aviram, M., and Rosenblat, M. (2004). Paraoxonases 1, 2, and 3, oxidative stress, and macrophage foam cell formation during atherosclerosis development. Free Radic Biol Med 37, 1304-1316.

Draganov, D. I., Stetson, P. L., Watson, C. E., Billecke, S. S., and La Du, B. N. (2000). Rabbit serum paraoxonase 3 (PON3) is a high density lipoprotein-associated lactonase and protects low density lipoprotein against oxidation. J Biol Chem 275, 33435-33442.

Galis, Z. S. (2004). Vulnerable plaque: the devil is in the details. Circulation 110, 244-246.

Gao, J., Wei, Y., Huang, Y., Liu, D., Liu, G., Wu, M., Wu, L., Zhang, Q., Zhang, Z., Zhang, R., et al. (2005). The expression of intact and mutant human apoAI/CIII/AIV/AV gene cluster in transgenic mice. J Biol Chem 280, 12559-12566.

Glass, C. K., and Witztum, J. L. (2001). Atherosclerosis. the road ahead. Cell 104, 503-516.

Harel, M., Aharoni, A., Gaidukov, L., Brumshtein, B., Khersonsky, O., Meged, R., Dvir, H., Rave R. B., McCarthy, A., Toker, L., et al. (2004). Structure and evolution of the serum paraoxonase family of detoxifying and anti-atherosclerotic enzymes. Nat Struct Mol Biol 11, 412-419.

Hedrick, C. C., Hassan, K., Hough, G. P., Yoo, J. H., Simzar, S., Quinto, C. R., Kim, S. M., Dooley, A., Langi, S., Hama, S. Y., et al. (2000). Short-term feeding of atherogenic diet to mice results in reduction of HDL and paraoxonase that may be mediated by an immune mechanism. Arterioscler Thromb Vasc Biol 20, 1946-1952.

Hegele, R. A., Connelly, P. W., Scherer, S. W., Hanley, A. J., Harris, S. B., Tsui, L. C., and Zinman, B. (1997). Paraoxonase-2 gene (PON2) G148 variant associated with elevated fasting plasma glucose in noninsulin-dependent diabetes mellitus. J Clin Endocrinol Metab 82, 3373-3377.

Jiang, Z. Y., Hunt, J. V., and Wolff, S. P. (1992). Ferrous ion oxidation in the presence of xylenol orange for detection of lipid hydroperoxide in low density lipoprotein. Anal Biochem 202, 384-389.

Lee, R. T., and Libby, P. (1997). The unstable atheroma. Arterioscler Thromb Vasc Biol 17, 1859-1867.

Leus, F. R., Zwart, M., Kastelein, J. J., and Voorbij, H. A. (2001). PON2 gene variants are associated with clinical manifestations of cardiovascular disease in familial hypercholesterolemia patients. Atherosclerosis 154, 641-649.

Libby, P. (2002). Inflammation in atherosclerosis. Nature 420, 868-874.

Lusis, A. J. (2000). Atherosclerosis. Nature 407, 233-241.

Mackness, M. I., Mackness, B., and Durrington, P. N. (2002). Paraoxonase and coronary heart disease. Atheroscler Suppl 3, 49-55.

Mackness, M. I., Mackness, B., Durrington, P. N., Fogelman, A. M., Berliner, J., Lusis, A. J., Navab, M., Shih, D., and Fonarow, G. C. (1998). Paraoxonase and coronary heart disease. Curr Opin Lipidol 9, 319-324.

Ng, C. J., Bourquard, N., Grijalva, V., Hama, S., Shih, D. M., Navab, M., Fogelman, A. M., Lusis, A. J., Young, S., and Reddy, S. T. (2006). Paraoxonase-2 deficiency aggravates atherosclerosis in mice despite lower apolipoprotein-B-containing lipoproteins: anti-atherogenic role for paraoxonase-2. J Biol Chem 281, 29491-29500.

Ng, C. J., Bourquard, N., Hama, S. Y., Shih, D., Grijalva, V. R., Navab, M., Fogelman, A. M., and Reddy, S. T. (2007). Adenovirus-mediated expression of human paraoxonase 3 protects against the progression of atherosclerosis in apolipoprotein E-deficient mice. Arterioscler Thromb Vase Biol 27, 1368-1374.

Ng, C. J., Shih, D. M., Hama, S. Y., Villa, N., Navab, M., and Reddy, S. T. (2005). The paraoxonase gene family and atherosclerosis. Free Radic Biol Med 38, 153-163.

Ng, C. J., Wadleigh, D. J., Gangopadhyay, A., Hama, S., Grijalva, V. R., Navab, M., Fogelman, A. M., and Reddy, S. T. (2001). Paraoxonase-2 is a ubiquitously expressed protein with antioxidant properties and is capable of preventing cell-mediated oxidative modification of low density lipoprotein. J Biol Chem 276, 44444-44449.

Ni, W., Egashira, K., Kitamoto, S., Kataoka, C., Koyanagi, M., Inoue, S., Imaizumi, K., Akiyama, C., Nishida, K. I., and Takeshita, A. (2001). New anti-monocyte chemoattractant protein-1 gene therapy attenuates atherosclerosis in apolipoprotein E-knockout mice. Circulation 103, 2096-2101.

Primo-Parmo, S. L., Sorenson, R. C., Teiber, J., and La Du, B. N. (1996). The human serum paraoxonase/arylesterase gene (PON1) is one member of a multigene family. Genomics 33, 498-507.

Reddy, S. T., Wadleigh, D. J., Grijalva, V., Ng, C., Hama, S., Gangopadhyay, A., Shih, D. M., Lusis, A. J., Navab, M., and Fogelman, A. M. (2001). Human paraoxonase-3 is an HDL-associated enzyme with biological activity similar to paraoxonase-1 protein but is not regulated by oxidized lipids. Arterioscler Thromb Vasc Biol 21, 542-547.

Schwartz, S. M., Galis, Z. S., Rosenfeld, M. E., and Falk, E. (2007). Plaque rupture in humans and mice. Arterioscler Thromb Vasc Biol 27, 705-713.

Shih, D. M., Gu, L., Hama, S., Xia, Y. R., Navab, M., Fogelman, A. M., and Lusis, A. J. (1996). Genetic-dietary regulation of serum paraoxonase expression and its role in atherogenesis in a mouse model. J Clin Invest 97, 1630-1639.

Shih, D. M., Gu, L., Xia, Y. R., Navab, M., Li, W. F., Hama, S., Castellani, L. W., Furlong, C. E., Costa, L. G., Fogelman, A. M., et al. (1998). Mice lacking serum paraoxonase are susceptible to organophosphate toxicity and atherosclerosis. Nature 394, 284-287.

Shih, D. M., Xia, Y. R., Wang, X. P., Miller, E., Castellani, L. W., Subbanagounder, G., Cheroutre, H., Faull, K. F., Berliner, J. A., Witztum, J. L., et al. (2000). Combined serum paraoxonase knockout/apolipoprotein E knockout mice exhibit increased lipoprotein oxidation and atherosclerosis. J Biol Chem 275, 17527-17535.

Shih, D. M., Xia, Y. R., Wang, X. P., Wang, S. S., Bourquard, N., Fogelman, A. M., Lusis, A. J., and Reddy, S. T. (2007). Decreased obesity and atherosclerosis in human paraoxonase 3 transgenic mice. Circ Res 100, 1200-1207.

Steinberg, D. (1997). Low density lipoprotein oxidation and its pathobiological significance. J Biol Chem 272, 20963-20966.

Tward, A., Xia, Y. R., Wang, X. P., Shi, Y. S., Park, C., Castellani, L. W., Lusis, A. J., and Shih, D. M. (2002). Decreased atherosclerotic lesion formation in human serum paraoxonase transgenic mice. Circulation 106, 484-490.

Watson, A. D., Berliner, J. A., Hama, S. Y., La Du, B. N., Faull, K. F., Fogelman, A. M., and Navab, M. (1995). Protective effect of high density lipoprotein associated paraoxonase. Inhibition of the biological activity of minimally oxidized low density lipoprotein. J Clin Invest 96, 2882-2891.

Watzinger, N., Schmidt, H., Schumacher, M., Schmidt, R., Eber, B., Fruhwald, F. M., Zweiker, R., Kostner, G. M., and Klein, W. (2002). Human paraoxonase 1 gene polymorphisms and the risk of coronary heart disease: a community-based study. Cardiology 98, 116-122.

1. Use of PON gene cluster in preparing the medicament for treating atherosclerosis in mammals.

2. The use according to claim 1, characterized in that the said mammal is selected from mouse or human.

3. The use according to claim 1, characterized in that the said mammal is human.

4. A method for the developing a PON gene cluster transgenic animal model, comprising the following steps of:
a) linearizing a vector comprising human PON gene cluster with an appropriate restrictive endonuclease; collecting and treating the vector DNA by conventional method for use in microinjection;
b) diluting the said DNA to approximate 1-2 ng/μL with buffer for microinjection and then microinjecting the diluted DNA into the fertilized egg of the animal surrogate;
c) placing the fertilized egg in M16 medium after being micro-injected, incubating the fertilized egg at 37° C. for 1-2 days;
d) transferring the fertilized egg of step c) to pseudo-pregnant animal surrogate, selecting PON gene cluster positive animals by PCR and Southern Blot Analysis after the newborn animals were delivered.

5. The method according to claim 4, characterized in that the said vector is a BAC vector RP11-104H16 and the said restrictive endonuclease is Not I.

6. The method according to claim 4, characterized in that the said animal is mouse.

7. The method according to claim 6, characterized in that the said fertilized egg is the fertilized egg of C57BL/6J mice.

8. Use of PON gene cluster in the development of PON gene cluster positive transgenic mice models with atherosclerosis.

9. The use according to claim 8, characterized in that the said transgenic mice models are obtained from the following steps of:
a) obtaining both PON gene cluster positive and apoE^{-/-} mice by crossing the mice obtained according to claim 7 and the apoE^{-/-} mice with atherosclerosis;
b) further crossing the mice obtained from a) with apoE^{-/-} mice for another generation and obtaining the mice with genotype both of PON gene cluster positive and apoE^{-/-} i.e., PON gene cluster positive transgenic mice models with atherosclerosis;
c) continuously crossing the mice obtained from b) with apoE^{-/-} mice to obtain large number of PON gene cluster positive transgenic mice models with atherosclerosis.

10. The method according to claim 5, characterized in that the said animal is mouse.

11. The method according to claim 10, characterized in that the said fertilized egg is the fertilized egg of C57BL/6J mice.


SEQUENCE LISTING (14 sequences)

SEQ ID NO: 1 - DNA, 22 bp, Artificial Sequence
  Description of Artificial Sequence Synthetic primer hPON1-RT-S
aaaggaatcgaaactggctctg

SEQ ID NO: 2 - DNA, 21 bp, Artificial Sequence
  Description of Artificial Sequence Synthetic primer hPON1-RT-A
gactgttggggttgaagctct

SEQ ID NO: 3 - DNA, 21 bp, Artificial Sequence
  Description of Artificial Sequence Synthetic primer hPON2-RT-S
ctcttcgtgtatgacccgaac

SEQ ID NO: 4 - DNA, 23 bp, Artificial Sequence
  Description of Artificial Sequence Synthetic primer hPON2-RT-A
acccattgttggcataaactgta

SEQ ID NO: 5 - DNA, 21 bp, Artificial Sequence
  Description of Artificial Sequence Synthetic primer hPON3-RT-S
aactttgcgccagatgaacca

SEQ ID NO: 6 - DNA, 22 bp, Artificial Sequence
  Description of Artificial Sequence Synthetic primer hPON3-RT-A
tcatgtggggatgattcacaac

SEQ ID NO: 7 - DNA, 22 bp, Artificial Sequence
  Description of Artificial Sequence Synthetic primer mPON1-RT-S
tactggtggtaaaccatccaga

SEQ ID NO: 8 - DNA, 23 bp, Artificial Sequence
  Description of Artificial Sequence Synthetic primer mPON1-RT-A
gcagctatatcgttgatgctagg

SEQ ID NO: 9 - DNA, 20 bp, Artificial Sequence
  Description of Artificial Sequence Synthetic primer mPON2-RT-S
gctctgagtttgctgggcat

SEQ ID NO: 10 - DNA, 20 bp, Artificial Sequence
  Description of Artificial Sequence Synthetic primer mPON2-RT-A
ccacgctaaagaaagccagg

SEQ ID NO: 11 - DNA, 19 bp, Artificial Sequence
  Description of Artificial Sequence Synthetic primer mPON3-RT-S
cctcactggacttccgtcg

SEQ ID NO: 12 - DNA, 23 bp, Artificial Sequence
  Description of Artificial Sequence Synthetic primer mPON3-RT-A
ggatcaacggtcaagttatccac

SEQ ID NO: 13 - DNA, 20 bp, Artificial Sequence
  Description of Artificial Sequence Synthetic primer beta-actin-s
gtggggcgccccaggcacca

SEQ ID NO: 14 - DNA, 24 bp, Artificial Sequence
  Description of Artificial Sequence Synthetic primer beta-actin-a
ctccttaatgtcacgcacgatttc